(19) United States
(12) Patent Application Publication
(10) Pub. No.: US 2007/0232998 A1
(43) Pub. Date: Oct. 4, 2007

(54) Injection Apparatus

(76) Inventors: Chang-Ming Yang, Junan Jen (TW); Chun-Mei Chou, Junan Jen (TW)

Correspondence Address: HDSL, 4331 STEVENS BATTLE LANE, FAIRFAX, VA, 22033, US

(21) Appl. No.: 11/760,731

(22) Filed: Jun. 8, 2007

Related U.S. Application Data
(63) Continuation-in-part of application No. 10/972,279, filed on Oct. 25, 2004, now abandoned.

(51) Int. Cl. A61M 5/50

(52) U.S. Cl. 604/110

(57) ABSTRACT

An injection apparatus includes an accommodating body, having a chamber formed therein, a needle retracting hole with a diameter smaller than the chamber and disposed at an end of the accommodating body and coupled to the chamber, a sliding path disposed on at least one side of the accommodating body, a first position defined on the sliding path and disposed proximate to the needle retracting hole, a slanting surface disposed on the sliding path and away from the needle retracting hole, and a second position defined on the slanting surface; and an injector, including a syringe head being smaller than the chamber and installed in the chamber, an injection needle disposed at an end of the syringe head and passing through the needle retracting hole, and a pushing portion disposed on at least one lateral side of the syringe head for sliding with respect to the sliding path; such that the pushing portion can be pushed from the first position to the second position along the sliding path, and the syringe head slides in the chamber to drive the injection needle to retract into the accommodating body, so as to produce a bias of the injection needle in the chamber.

### RELATED APPLICATIONS

This application is a continuation-in-part of U.S. patent application Ser. No. 10/972,279, filed on Oct. 25, 2004.

### FIELD OF THE INVENTION

The present invention relates to an injection apparatus, and more particularly to an injection apparatus having a needle that can be retracted and stored safely to assure a single use of the needle and preclude users from reusing the needle by accident.

### BACKGROUND OF THE INVENTION

According to the statistics provided by the World Health Organization (WHO), a large percentage of injection apparatuses being reused in different countries over the world creates a high risk and greatly increases the chance of spreading infectious diseases, and most of the reuses usually occur in developing countries and undeveloped countries. The WHO reports also show that 8˜16 million people are infected by hepatitis B virus (HBV), 2˜4.5 million people are infected by hepatitis C virus (HCV), and 7,000˜15,000 people are infected by Human Immunodeficiency Virus/Acquired Immunodeficiency Syndrome (HIV/AIDS) all over the world each year.

In the past, injection apparatuses were recyclable and reusable, but they must be sterilized before their reuse. Unfortunately, the sterilization is usually incomplete, and incomplete sterilizations may induce infections easily. According to the present medical standard operation procedure, the reuse of an injection apparatus is not allowed, mainly for avoiding cross infections.

At present, a conventional injection apparatus generally includes a syringe barrel, a plunger disposed at an end of the syringe barrel, a syringe head sheathed onto another end of the syringe barrel, and an injection needle disposed at an end of the syringe head. However, the foregoing structure still has the following drawbacks in its practical application: 1. Since the injection needle looks like a new one after its use, and it is difficult for users to distinguish whether or not the injection needle has been used, therefore the injection needle may be reused by accident, and that will induce cross infections and spread infectious diseases. 2. After an injection performed by an injection apparatus is completed, medical professionals may touch a patient's body fluids while withdrawing the injection needle and may be pierced and injured by the exposed tip of the injection needle, and thus creating a high risk to medical professionals. In fact, many needle stick injuries or infections can be prevented in advance. In view of the aforementioned drawbacks, health authorities of each country set forth related regulations and standards to control blood infectious diseases, in hope of establishing a safety management for the injection apparatuses and related medical equipments and materials, and demanding a safer design for an injection apparatus.

In view of the foregoing shortcomings of the prior art, the inventor of the present invention based on years of experience in the related industry to conduct extensive researches and experiments, and finally developed a injection apparatus in accordance with the present invention to overcome the shortcomings of the prior art.

### SUMMARY OF THE INVENTION

The present invention is to provide an injection apparatus to assure a single use of a needle that can be retracted and stored safely to preclude reusing the needle by accident.

The present invention provides an injection apparatus, comprising: an accommodating body, having a chamber formed therein, a needle retracting hole with a diameter smaller than the chamber and disposed at an end of the accommodating body and coupled to the chamber, a sliding path disposed on at least one side of the accommodating body, a first position defined on the sliding path and disposed proximate to the needle retracting hole, a slanting surface disposed on the sliding path and away from the needle retracting hole, and a second position defined on the slanting surface; and an injector, including a syringe head being smaller than the chamber and installed in the chamber, an injection needle disposed at an end of the syringe head and passing through the needle retracting hole, and a pushing portion disposed on at least one lateral side of the syringe head for sliding with respect to the sliding path; such that the pushing portion can be pushed from the first position to the second position along the sliding path, and the syringe head slides in the chamber to drive the injection needle to retract into the accommodating body, so as to produce a bias of the injection needle in the chamber.

In the foregoing structure, the pushing portion is pushed from the first position to the second position along the sliding path, so that the injection needle is retracted from the needle retracting hole into the accommodating body. Since the second position is situated on a slanting surface and there exists a different interval, therefore the syringe head is shifted, and the injection needle in the chamber is biased with respect to the needle retracting hole. As a result, the syringe head no longer can enter into the needle retracting hole again.

To make it easier for our examiner to understand the objective of the invention, its structure, innovative features, and performance, we use preferred embodiments together with the attached drawings for the detailed description of the invention.

### BRIEF DESCRIPTION OF THE DRAWING

FIG. 1 is a perspective view of an injection apparatus in accordance with the present invention;

FIG. 2 is an exploded view of an injection apparatus in accordance with the present invention;

FIG. 3 is a partial section view of an injection apparatus in accordance with a first preferred embodiment of the present invention;

FIG. 4 is a schematic view of a first application of an injection apparatus in accordance with a first preferred embodiment of the present invention;

FIG. 5 is a schematic view of a second application of an injection apparatus in accordance with a first preferred embodiment of the present invention;

FIG. 6 is a schematic view of an application of an injection apparatus in accordance with a second preferred embodiment of the present invention;

FIG. 7 is a perspective view of an injection apparatus in accordance with a third preferred embodiment of the present invention;

FIG. 8 is an exploded view of an injection apparatus in accordance with a third preferred embodiment of the present invention;

FIG. 9 is a schematic view of a first application of an injection apparatus in accordance with a third preferred embodiment of the present invention;

FIG. 10 is a schematic view of a second application of an injection apparatus in accordance with a third preferred embodiment of the present invention;

FIG. 11 is a schematic view of a first application of an injection apparatus in accordance with a fourth preferred embodiment of the present invention; and

FIG. 12 is a schematic view of a second application of an injection apparatus in accordance with a fourth preferred embodiment of the present invention.

### DETAILED DESCRIPTION OF THE PREFERRED EMBODIMENTS

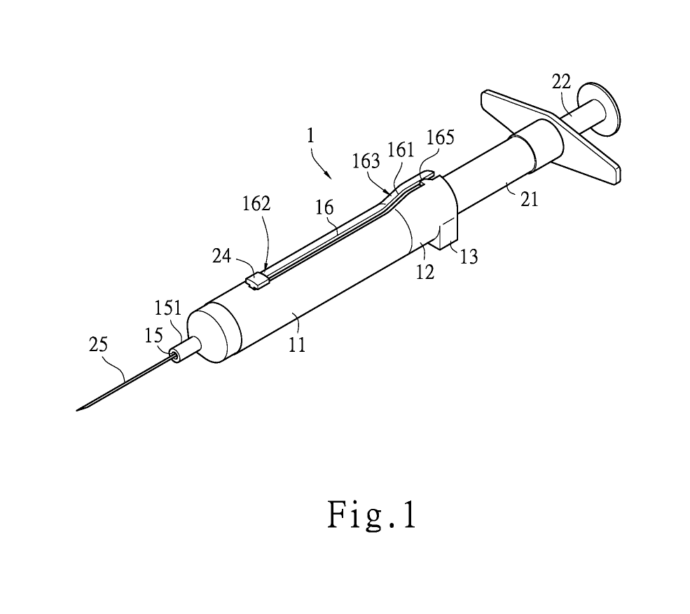

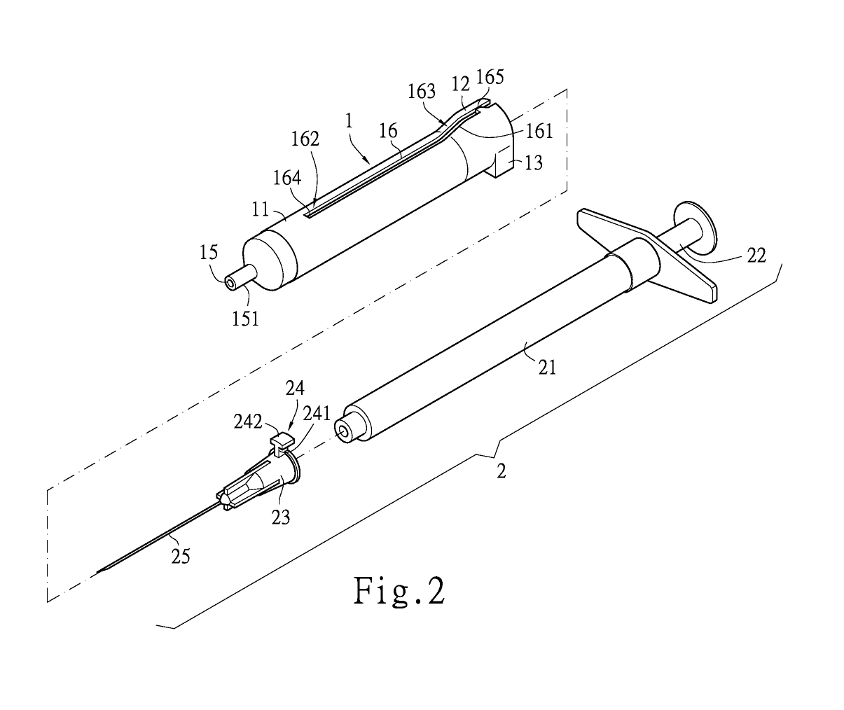

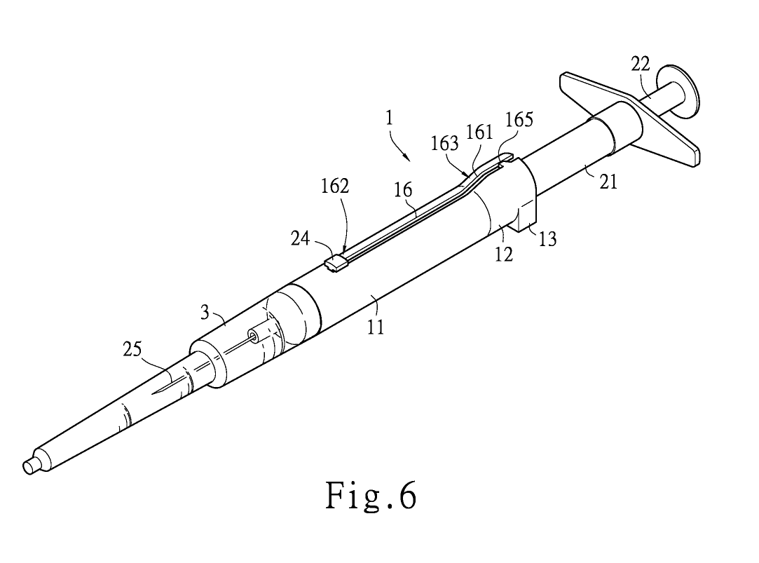

Referring to FIGS. 1, 2, 3 and 6, an injection apparatus of the present invention comprises: an accommodating body 1, an injector 2 and a needle cover 3, wherein the accommodating body 1 includes a narrow tube 11 and a wide tube 12 extended from a rear end of the narrow tube 11 and forms a slanting surface 161, and the accommodating body 1 includes at least one base 13 on an external side of the wide tube 12, a chamber 14 formed in and interconnected with the narrow tube 11 and the wide tube 12 of the accommodating body 1, a needle retracting hole 15 disposed at a front end of the narrow tube 11 of the accommodating body 1 and away from the wide tube 12 and having a diameter smaller than the chamber 14 and coupled to the chamber 14, a stop wall 141 formed around the periphery of the needle retracting hole 15 of the chamber 14, and a needle tube 151 extended outwardly from the needle retracting hole 15 of the accommodating body 1.

The accommodating body 1 has a sliding path, and the sliding path has a sliding slot 16 disposed on a surface of the accommodating body 1 and extended from a surface of the narrow tube 11 to a surface of the wide tube 12, and a rear end of the sliding slot 16 is extended to a rear end of the wide tube 12 of the accommodating body 1.

A first position 162 disposed at the narrow tube 11 and a second position 163 disposed at a curved section 161 are defined as positions on the sliding slot 16 that are proximate to and away from the needle retracting hole 15 respectively, and the sliding slot 16 includes a first stop portion 164 disposed at the first position 162 composed of a stop wall and a second stop portion 165 composed of an elastic protruding arm and disposed on a lateral side of the second position 163 of the sliding slot 16.

The injector 2 includes a syringe barrel 21 sheathed into an end of the accommodating body 1, a plunger 22 installed to an end of the syringe barrel 21, and a syringe head 23 coupled to another end of the syringe barrel 21. The syringe head 23 is smaller than the chamber 14 and disposed in the chamber 14, and an end of the syringe head 23 has an injection needle 25 passing through the needle retracting hole 15 and the needle tube 151, and the syringe head 23 includes a pushing portion 24 disposed on at least one side of the syringe head 23 for sliding with respect to the sliding slot 16, and the pushing portion 24 includes a neck portion 241 disposed on a lateral end of the syringe head 23 and protruding through the sliding slot 16 and a pushing member 242 disposed at an end of the neck portion 241.

When the injection apparatus is in use, the needle cover 3 is sheathed onto the narrow tube 11 of the accommodating body 1 for accommodating the injection needle 25. Further, the pushing member 242 of the pushing portion 24 is pushed to move from the first position 162 to the second position 163 along the sliding path, such that the syringe head 23 can slide in the chamber 14 to drive the injection needle 25 to retract from the needle retracting hole 15 into the accommodating body 1.

During installation, the neck portion 241 of the pushing portion 24 is placed into the sliding slot 16 along a rear end of the wide tube 12 and passed through the second stop portion 165, so that the syringe head 23 is installed in the chamber 14, and the injection needle 25 is passed through the needle retracting hole 15 and the needle tube 151.

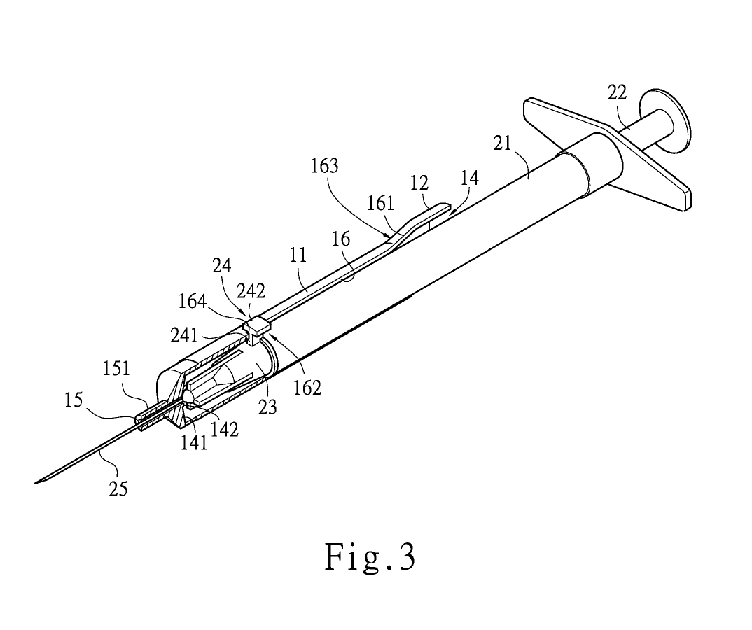

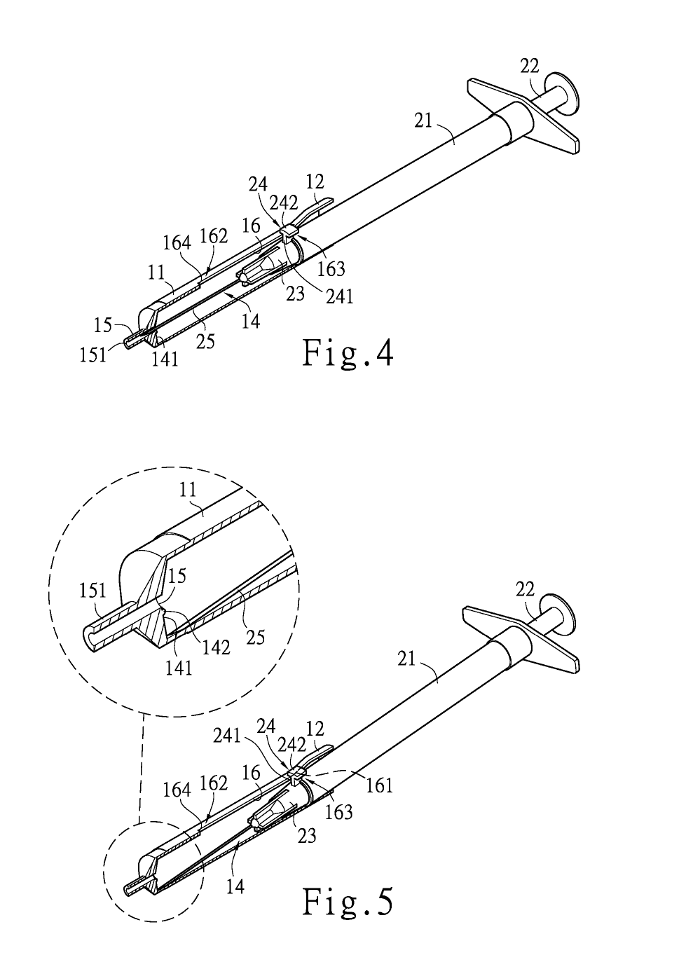

Referring to FIGS. 3 to 5 for the aforementioned structure, after a medical professional completes an injection procedure, the pushing portion 24 is pushed to move from the first position 162 to the second position 163 along the sliding path, such that the syringe head 23 is shifted to drive the injection needle 25 to retract from the needle retracting hole 15 into the accommodating body 1, while synchronously producing a bias. The injection needle 25 in the chamber 14 is biased with respect to the needle retracting hole 15, so that the injection needle 25 abuts the stop wall 141 around the needle retracting hole 15 in the chamber 14 and no longer can enter into the needle retracting hole 15 again, so as to preclude a reuse of the injection needle 25. Further, the accommodating body 1 has a base 13 on an external side of the wide tube 12, such that the accommodating body 1 can be placed on a tabletop or any platform.

Referring to FIG. 5 for the aforementioned structure, the stop wall 141 around the needle retracting hole 15 includes at least one protruding dot 142.

When the injection needle 25 of the aforementioned structure is retracted from the needle retracting hole 15 into the accommodating body 1, the injection needle 25 is stopped by the protruding dot 142 disposed on the stop wall 141 around the needle retracting hole 15 in the chamber 14 to prevent the injection needle 25 from entering into the needle retracting hole 15 again.

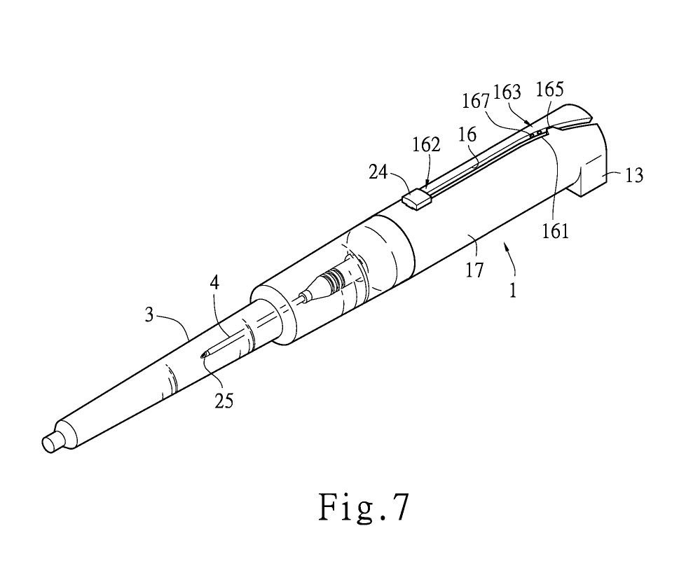

Referring to FIG. 7 for a second preferred embodiment of the present invention, the needle tube 151 is sheathed into a Catheter 4, and the injection needle 25 is sheathed into the Catheter 4.

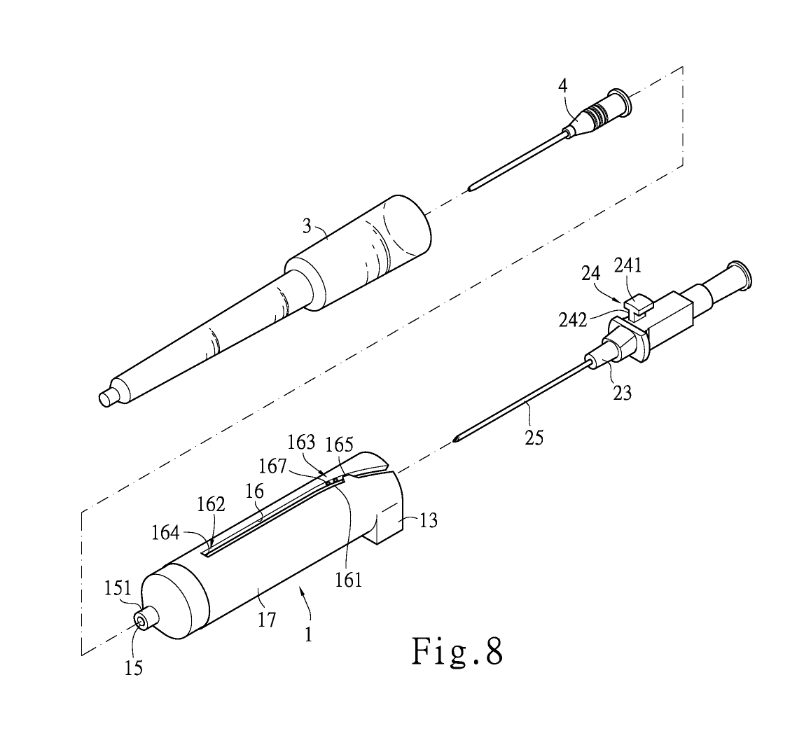

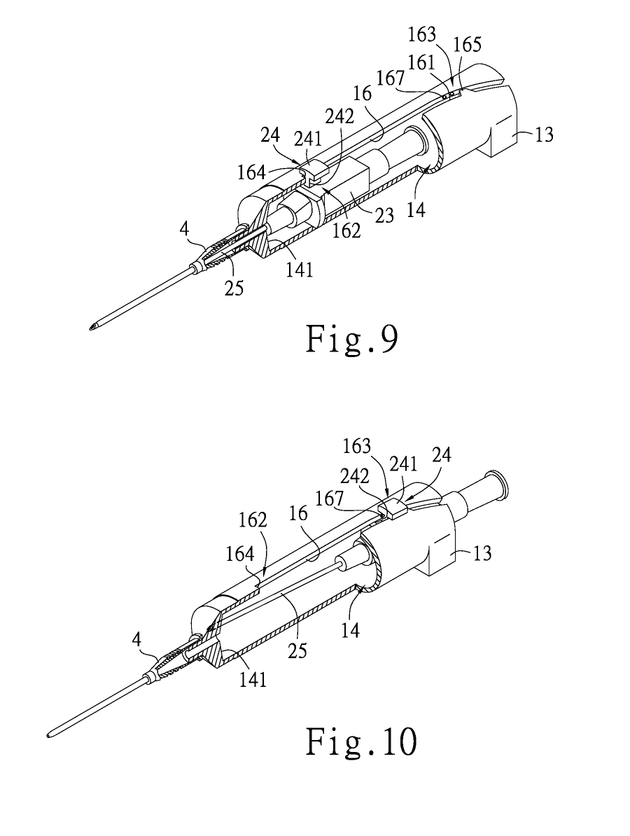

Referring to FIG. 7 to 10 for a third preferred embodiment of the present invention, the accommodating body 1 of the aforementioned structure has a width equal to that of a barrel 17, and the chamber 14 is formed inside the barrel 17, and a rear end of the sliding slot 16 forms a curved section 161 along a lateral surface of the barrel 17. There are protrusions 167 formed on the curved section 161.

After the injection needle 25 is inserted into a human body, and the Catheter 4 is entered into a human body, medical professionals can push the pushing portion 24 to move from the first position 162 to the second position 163 along the sliding path, so that the syringe head 23 is shifted to drive the injection needle 25 to retract from the needle retracting hole 15 into the accommodating body 1 and separate from the Catheter 4, and the Catheter 4 can remain in the human body.

After a medical professional completes an injection procedure, the pushing portion 24 is pushed to move from the first position 162 to the second position 163 along the sliding path, such that the syringe head 23 is shifted to drive the injection needle 25 to retract from the needle retracting hole 15 into the accommodating body 1 while synchronously producing a bias, and the injection needle 25 in the chamber 14 is biased with respect to the needle retracting hole 15 and abuts the stop wall 141 around the needle retracting hole 15 in the chamber 14, and the injection needle 25 can no longer enter into the needle retracting hole 15 again. When the pushing portion 24 is pushed to the second position 163, the protrusions 167 on the curved section 161 increase the friction between the curved section 161 and the neck portion 241 to further ensure the injection structure can not be reused.

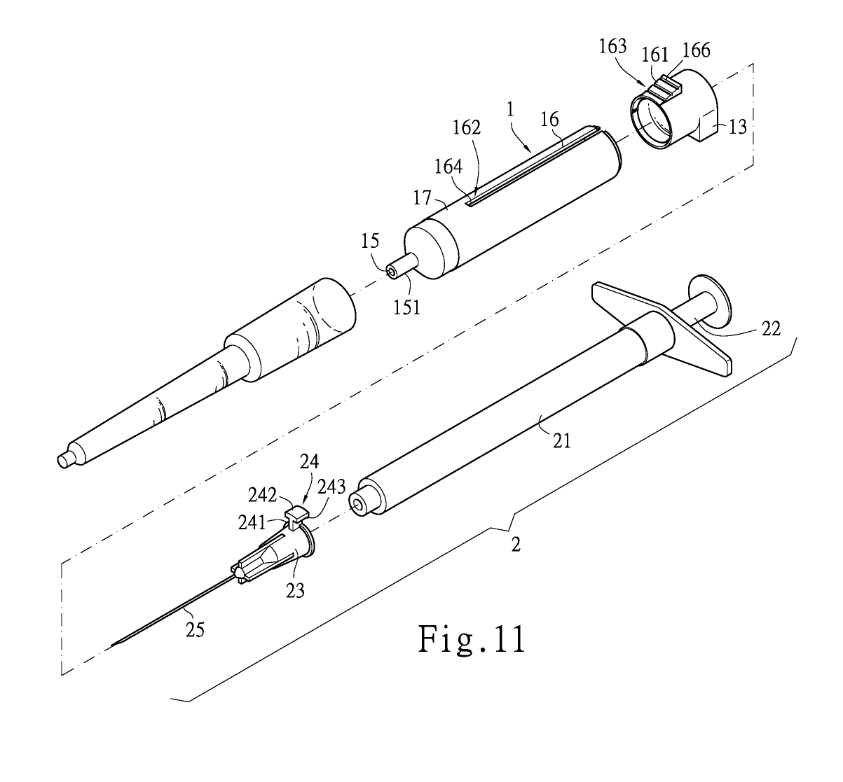

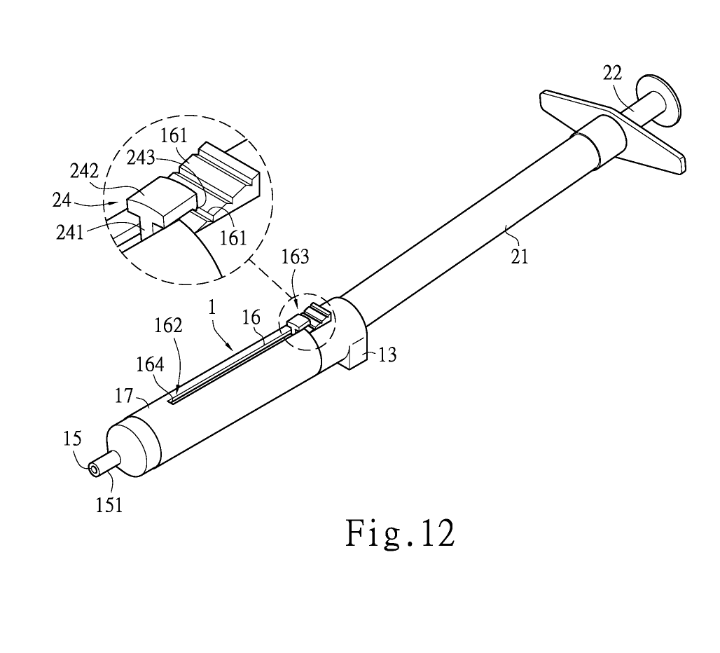

Referring to FIGS. 11 and 12 for a fourth preferred embodiment of the present invention, the accommodating body 1 of the aforementioned structure has a width equal to that of the barrel 17, and a lateral side of the accommodating body 1 has a sliding path extended from a surface of the accommodating body 1 to a rear end of the barrel 17, and an end of the barrel 17 has a base portion 18 for sheathing the accommodating body 1 into the base portion 18, and a side wall of the base portion 18 has a slanting surface 161 for allowing the pushing portion 24 of the injector to be pressed onto the slanting surface 161. Further, the slanting surface 161 has a groove 166 corresponding to a protruding member 243 on the pushing member 242 for fixing the pushing portion 24 onto the slanting surface.

To facilitate the installation of the aforementioned structure, the injector 2 inserts an injection needle 25 before it is installed into the accommodating body 1, and the accommodating body 1 is sheathed onto base portion 18. After a medical professional completes an injection procedure, the pushing portion 24 is pushed to move from the first position 162 to the second position 163 along the sliding path, such that the syringe head 23 is shifted to drive the injection needle 25 to retract from the needle retracting hole 15 into the accommodating body 1 while synchronously producing a bias, and the injection needle 25 in the chamber 14 is biased with respect to the needle retracting hole 15 and the injection needle 25 abuts the stop wall 141 around the needle retracting hole 15 in the chamber 14, and thus the injection needle 25 can no longer enter into the needle retracting hole 15 again. Since the protruding member 243 on the pushing member 242 is latched with a groove 166 on the slanting surface 161, a reuse of the injector can be prevented.

In summation of the description above, the present invention has the following advantages: 1. In the present invention, the pushing portion 24 is pushed to move from the first position 162 to the second position 163 along the sliding path, so that the injection needle 25 is retracted into the chamber 14 and biased with respect to the needle retracting hole 15, and the injection needle 25 abuts the stop wall 141 around the needle retracting hole 15 in the chamber 14, so as to prevent the injection apparatus from being reused by accident, preclude the possibility of cross infections and spreads of infectious diseases, and improve the safety of injections. 2. In the present invention, when a medical professional removes the injection apparatus, the pushing portion 24 is pushed to move from the first position 162 to the second position 163 along the sliding path, so that the injection needle 25 is retracted into the chamber 14 after an injection and produces a bias with respect to the needle retracting hole 15. The injection needle 25 abuts the stop wall 141 around the needle retracting hole 15 in the chamber 14 to prevent medical professionals from being pierced or injured by the injection needle 25.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. An injection apparatus, comprising:
an accommodating body, forming a chamber therein, a needle retracting hole with a diameter smaller than the chamber and disposed at an end of the accommodating body and coupled to the chamber, a sliding path disposed on at least one side of the accommodating body, a first position defined at the sliding path and proximate to the needle retracting hole, a slanting surface disposed on the sliding path and away from the needle retracting hole, and a second position defined on the slanting surface; and
an injector, including a syringe head being smaller than the chamber and disposed in the chamber, an injection needle disposed at an end of the syringe head and passing through the needle retracting hole, and a pushing portion disposed on at least one side of the syringe head for sliding with respect to the sliding path;
such that the pushing portion can be pushed from the first position to the second position along the sliding path, and the syringe head slides in chamber to drive the injection needle to retract from the needle retracting hole into the accommodating body, so as to produce a bias of the injection needle in the chamber.

2. The injection apparatus as recited in claim 1, wherein the sliding path includes a sliding slot created on a surface of the accommodating body.

3. The injection apparatus as recited in claim 2, wherein the sliding slot is extended at least to an end of the accommodating body.

4. The injection apparatus as recited in claim 3, wherein the sliding slot includes at least one stop portion.

5. The injection apparatus as recited in claim 4, wherein the stop portion forms a stopping member on a lateral side of the sliding slot.

6. The injection apparatus as recited in claim 4, wherein the stop portion is situated at the first position.

7. The injection apparatus as recited in claim 6, wherein the accommodating body includes a narrow tube, a slanting surface extended from an end of the narrow tube to a wide tube, and the sliding slot is extended from a surface of the narrow tube to a surface of the wide tube.

8. The injection apparatus as recited in claim 7, wherein the slanting surface is situated at the second position.

9. The injection apparatus as recited in claim 7, wherein the needle retracting hole is disposed at another end of the narrow tube and away from the wide tube.

10. The injection apparatus as recited in claim 2, wherein the pushing portion includes a neck portion disposed on a lateral end of the syringe head and protruding through the sliding slot, and a pushing member disposed at an end of the neck portion.

11. The injection apparatus as recited in claim 7, further comprising at least one protrusion formed on the slanting surface.

12. The injection apparatus as recited in claim 1, wherein the chamber forms a stop wall at the periphery of the needle retracting hole.

13. The injection apparatus as recited in claim 12, wherein the stop wall at the periphery of the needle retracting hole includes at least one stop member.

14. The injection apparatus as recited in claim 6, wherein accommodating body includes a base portion disposed at an end of the accommodating body for sheathing the accommodating body into the base portion.

15. The injection apparatus as recited in claim 14, wherein the base portion include at least one slanting surface on a sidewall of the base portion, for allowing the pushing portion of the injector to be pushed onto the slanting surface.

16. The injection apparatus as recited in claim 15, wherein the slanting surface includes at least one groove.

17. The injection apparatus as recited in claim 10, wherein the pushing member comprises a protruding member latched with the corresponding groove of the slanting surface for fixing the pushing portion.

18. The injection apparatus as recited in claim 1, wherein the accommodating body includes at least one base disposed on an external side of the accommodating body.

19. The injection apparatus as recited in claim 1, further comprising a needle cover sheathed onto the accommodating body for accommodating the injection needle.

20. The injection apparatus as recited in claim 1, wherein the accommodating body includes a needle tube extended outwardly from the needle retracting hole of the accommodating body for passing the injection needle.

21. The injection apparatus as recited in claim 20, wherein the needle tube is connected to a Catheter, and the injection needle is passed into the Catheter.

22. The injection apparatus as recited in claim 1, wherein the injector includes a syringe barrel, and a plunger disposed at an end of the syringe barrel, and another end of the syringe barrel is coupled to the syringe head.

23. The injection apparatus as recited in claim 22, wherein the accommodating body has an end provided for coupling the syringe barrel.
